(19) United States
(12) Patent Application Publication
(10) Pub. No.: US 2026/0157402 A1
(43) Pub. Date: Jun. 11, 2026

(54) ADDITIVE FOR RUMINANT ANIMAL NUTRITION AND FEED COMPRISING THE SAME

(71) Applicant: ZORION US LLC, Cumming, GA (US)

(72) Inventors: Fernando Javier Maguregui, Santa Fe (AR); Fernando Rubén Ceferino Barra, Santa Fe (AR); Lucas De Souza Rigolin, Maringá (BR)

(73) Assignee: ZORION US LLC, Cumming, GA (US)

(21) Appl. No.: 19/126,208

(22) PCT Filed: Nov. 3, 2023
(86) PCT No.: PCT/US2023/078697

§ 371 Date: Apr. 30, 2025

Related U.S. Application Data
(60) Provisional application No. 63/422,183, filed on Nov. 3, 2022.

(51) Int. Cl. A23K 50/10; A23K 10/30; A23K 20/22; A23K 20/24; A23K 20/28; A61K 9/00; A61K 33/04; A61K 33/06; A61K 33/08; A61K 47/02; A61P 3/02

(52) U.S. Cl. CPC A23K 50/10; A23K 10/30; A23K 20/22; A23K 20/24; A23K 20/28; A61K 9/0056; A61K 33/04; A61K 33/06; A61K 33/08; A61K 47/02; A61P 3/02

(57) ABSTRACT

An additive for ruminant animal nutrition, comprising the following components: sodium hydroxide, calcium oxide, calcium hydroxide and sodium sulfate, along with an inert food-grade excipient, wherein said additive is to be added to ruminant animal feed in such a way that the pH of the feed is greater than 9. Ruminant feed comprising said additive for animal nutrition.

### FIELD OF THE INVENTION

The present invention relates to the field of livestock feed, particularly for ruminant livestock, and more particularly to additives used in ruminant nutrition.

### PRIOR ART INFORMATION

Over thousands of years, ruminant animals have evolved to adapt to their environment. To that effect, they have developed a digestive system with four stomachs that allows them to quickly harvest food from grasslands and then retreat to begin the digestion process through rumination—the process of regurgitating and re-chewing the regurgitated bolus—which gives a significant group of animals the name “ruminants”. Alongside the ruminants, additionally, a large number of microorganisms have evolved and adapted, including hundreds of colonies of bacteria, yeasts, fungi, and protozoa specialized in digesting grass.

This complex ecosystem that lives inside the ruminants' stomachs ensures the passage of microorganisms from mother to offspring through two implantation mechanisms. One is through fecal matter during birth, and the other is through a physiological mechanism specific to ruminants, where they inhale ruminal air that reaches the larynx during regurgitation. Additionally, female ruminants implant microorganisms from their pre-stomachs by licking the calf for hygiene purposes after birth.

In the following diagrams, significant differences between human digestion and ruminant digestion can be observed, with the complexity of the latter being evident at first glance.

These diagrams were taken from Van Soest P. J. (1994).

The ecosystem that has developed over years of evolution within the pre-stomachs of ruminant animals is a facultative anaerobic system where the microorganisms (MO) have limited available oxygen. If the diet is predominantly pastoral, i.e. herbivorous, the pH is very close to neutrality or slightly alkaline (see FIG. 1). Over the years of evolution, ruminants have acquired MO capable of producing volatile fatty acids (VFAs) that acidify the environment. In this way, they generate the majority of the energy needed for living and regulating the acidity of the pre-stomachs by utilizing carbohydrates as a method of adenosine triphosphate (ATP) production, along with the concomitant production of microbial cells that become the ruminant's food. This oversimplified explanation must be considered bearing in mind that ruminants have been evolving for thousands of years.

In recent years, humans have needed to increase production efficiency, so they started adding carbohydrate grains and proteins to the diet. This increased production by improving ration digestibility but also modified the rumen, shifting it from a neutral or slightly alkaline ecosystem to a predominantly acidic one (see FIG. 2). This change has led to disorders in the ruminant's digestive system, resulting in a different fermentation process that produces a higher proportion of lactate and fewer volatile fatty acids (VFAs), at the expense of the animals' health, mainly due to acidosis.

Acidosis in herbivores occurs when the anaerobic and facultative anaerobic microorganisms in the rumen and cecum ferment carbohydrates into VFAs and lactates. When the supply of carbohydrates increases abruptly, i.e. after grain overload or during adaptation to high-concentrate diets, the lactate concentration rapidly increases in relation to the total acids normally present in the digestive tract at low concentrations. Dunlop and Hammond (1965) coined the term “lactic acidosis” for this metabolic alteration and attributed numerous diseases to this decrease in pH.

Lean and Golder (2019); Owens et al. (1998), and Kleen et al. (2003) are just some of the many authors who have described the genesis of this metabolic disorder in ruminants and the associated diseases for both (i) clinical acidosis and (ii) subacute ruminal acidosis (SARA). Both clinical lactic acidosis and subacute acidosis manifest with decreased feed intake, loss of rumen motility, diarrhea, poor daily weight gains, resulting in a decline in body condition, abomasal torsion, hypocalcemia, loss of milk production, depression of butyric fat, production of toxins that influence inflammatory processes and the population of ruminal microorganisms, compromising the epithelia of the forestomachs, leading to ruminal epithelial parakeratosis, pododermatitis, lung infections and hepatic abscesses, increased passage rate of ingesta through the digestive tract, malabsorption syndrome, increased osmolarity or osmotic concentration, which is the measurement of solute concentration defined as the number of osmoles (Osm) of a solute per liter (L) of rumen solution, high culling rate of animals, etc.

Animals coexist with SARA due to the generation of excessive volatile fatty acids (VFAs) in the rumen (Nagaraja et al., 2007). Since the 1950s, producers have started incorporating buffers as a tool, and since the 1970s, companies have begun developing additives such as antibiotics, ionophores, yeasts, probiotics, etc., to make fermentation more efficient and to ensure that the pH decrease is as controlled as possible, due to the need to incorporate large amounts of carbohydrates into the diet, especially when the carbohydrates are rapidly fermentable (RFCH) (Lean et al., 2018).

Now, field technicians, as described by Lean I. J. et al. 2018, have a range of products available to address this metabolic disorder, such as: i) buffers to bring the rumen pH close to the chosen buffer's pK, such as sodium bicarbonate, potassium carbonate, potassium bicarbonate, sodium sesquicarbonate, etc.; ii) antibiotics, the combination of ionophores with antibiotics is common, such as tylosin plus monensin and/or virginiamycin, favomycin (bambermycin) plus monensin being the most studied combinations, among others; iii) ionophores, such as monensin, lasalocid, salinomycin, virginiamycin, and narasin, which are the most commonly used ionophore antibiotics in animal production, all of which work by targeting certain colonies of bacteria in the rumen-cecum to decrease lactate production and alter the proportions of volatile fatty acids (VFAs), often leading to decreased feed intake by animals; iv) yeasts, which stabilize rumen functions, increase pH, reduce lactate, improve fiber digestion, and have a slight improvement in VFA production (Li et al. 2016), but most importantly, they enhance immune response within the digestive system (Bach et al. 2018); and finally, v) probiotics, for which there is also evidence of promoting benefits in acidosis control.

In summary, if we take a look at the main scientific dissemination journals today, we can observe that numerous publications aimed at manipulating the rumen environment and minimizing acidosis correspond to buffering substances or buffers, or others that modify metabolic activity and the proportion of certain microorganisms such as ionophores, or that improve the rumen environment such as microbial cultures or enzymes that increase food utilization, to name the most important ones.

The present invention is based on the use of a new concept of rumen balance that respects the physiology, ecology, and safety of the raw materials used. It consists of providing the ruminant with a complete diet with a pH (potential of hydrogen), which is a measure of the degree of acidity or alkalinity of a substance or solution, of nine or higher. This way, the forestomachs need to continuously acidify the rumen through the production of volatile fatty acids (see FIG. 3), and these fulfill the same function in ruminants as in pasture diets, using alkalizing agents to balance energy and OH (hydroxide ions) in order to optimize each of the functions of the ruminal ecosystem. Additionally, it prevents lactic acidosis and, in this way, maximizes the production of volatile fatty acids and microbial protein, utilizing the rumen as it was selected by nature and humans to achieve a healthy animal that converts food into milk or meat more efficiently.

In the study by Tripathi et al. (2004), it is observed that the dry matter intake in lambs with different inclusions of sodium bicarbonate follows a quadratic distribution, meaning that intake increases as the buffer inclusion increases, but beyond a certain point, dry matter intake decreases. Blood glucose production, on the other hand, showed a linear distribution and consistently increased.

FIG. 4 shows the solubility profile of soy protein isolate as a function of pH (Sarai Serrano Vallecios, M., 2015), where it can be observed that the protein solubility is maximized at a pH greater than six.

Sherasia et al. (2015) demonstrate that an improvement in feed efficiency leads to a reduction in methane emissions from the animal. Moss et al. (2000) indicate that the main pathway for methane production by methanogenic bacteria is 4H₂+CO₂→CH₄+2 H₂O, which is necessary to remove the protons generated by the production of volatile fatty acids in the rumen.

Therefore, it is desirable to have an additive that allows for an improvement in conversion efficiency, i.e, the ratio between food consumed and meat, milk or wool produced, accompanied by a significant decrease in greenhouse gas emissions, that is palatable and accepted by animals, that does not require adaptation to a change in diet and animals do not present health problems.

### SUMMARY OF THE INVENTION

Thus, in order to solve the drawbacks of prior art, an object of the present invention is an additive for ruminant animal nutrition comprising the following components: sodium hydroxide, calcium oxide, calcium hydroxide and sodium sulfate, such that said components are present in the following proportions expressed in % w/w:

| | | Inclusion of | | |
|---|---|---|---|---|
| | | components in | | |
| | | the invention | | Additive in TDD |
| Component in solid form | | % min. | % max. | (in AFB) |
| NaOH | Sodium | 25 | 69 | An effective amount |
| | hydroxide | | | necessary for the pH |
| CaO | Calcium oxide | 0 | 31 | of the TDDEF to be |
| Ca(OH)₂ | Calcium | 0 | 50 | greater than 9 |
| | hydroxide | | | |
| Na₂SO₄ | Sodium sulfate | 0 | 19 | |

| | |
|---|---|
| An inert excipient that is | Q.S. to reach |
| food-grade acceptable | 100% w/w |

wherein Q.S. stands for quantum satis (sufficient quantity);
wherein TDD stands for “total daily diet”, which is the sum of the different rations that an animal ingests in a day;
wherein TDDEF stands for “total daily diet excluding forage”; and
wherein AFB stands for “as-fed basis” which is the ration including water expressed as moisture.

Preferably, the inert food-grade excipient is selected from the group consisting of shell, silicates, limestone, aluminosilicate, and mixtures thereof.

Particularly, the aluminosilicate excipient is selected from the group consisting of zeolite, bentonite, diatomaceous earth, kaolin, and mixtures thereof.

Specifically, each component of the additive is considered a pure substance in its formulation.

Distinctively, the heat contribution of the additive, resulting from the combination of dilution heat and reaction heat, consists of an amount ranging from 1.5 to 5.0 kcal per kilogram of TDDEF.

Especially, the calorific contribution from dilution heat is originated from NaOH and Na₂SO₄.

Also especially, the calorific contribution from reaction heat is originated from CaO and H₂O.

Substantially, the contribution of hydroxyl groups (OH) per kilogram of TDDEF provided by the additive is between 5.5 and 8.5 grams of OH.

Specifically, the contribution of hydroxyl groups (OH) to TDDEF is achieved through any of the following: additive formulation, reaction of CaO with H₂O present in TDDEF, during the food preparation process with the additive, ruminal digestive system, and their combinations.

Primarily, the food is a regular diet consisting of feed ingredients for ruminants.

Also primarily, the diet consists of balanced rations composed of concentrated feed, forages, minerals, food additives, and mixtures thereof in the form of a total mixed ration (TMR) or TDD.

Another embodiment of the present invention is a ruminant feed comprising an effective amount of the described animal nutrition additive, wherein said effective amount is necessary for the pH of TDDEF to be greater than 9.

Preferably, the feed consists of a regular diet consisting of feed ingredients for ruminants.

More preferably, the diet consists of balanced rations composed of concentrated feed, forages, minerals, food additives, and mixtures thereof in the form of a TMR or TDD.

Even more preferably, the concentrated feeds are selected from corn, sorghum, barley, wheat, oats, wheat middlings, soybean hulls, corn semolina, gluten, malt, dried distillers grains (DDG), glycerol, whey, whey permeate, molasses, beef tallow, blood meal, vegetable fat, animal fat, vegetable protein, animal protein, soybean meal, extruded soybean meal, soybean pellets, cereal meals, and mixtures thereof.

Also, more preferably, the forages are selected from hay, straw, pasture, grass, cobs, cotton hulls, corn stalks, and mixtures thereof.

Furthermore, more preferably, the minerals are selected from sodium, potassium, magnesium, calcium, phosphorus, sulfur, manganese, selenium, chromium, cobalt, iodine, iron, zinc, copper, and mixtures thereof.

Even more preferably, the food additives comprise technological additives selected from adsorbents, binders, anti-caking agents, antioxidants, anti-humectants, preservatives, emulsifiers, stabilizers, thickeners, gelling agents, acidity regulators, humectants, and mixtures thereof; sensory additives selected from colorants, pigments, flavorings, palatability enhancers, and mixtures thereof; nutritional additives, vitamins, trace elements, organic micro-minerals, amino acids, peptides, urea, urea derivatives, and mixtures thereof; zootechnical additives selected from enzymes, probiotics, prebiotics, postbiotics, acidifiers, plant extracts, antioxidants, and mixtures thereof; and antimicrobial additives selected from ionophores, antibiotics, hormones, and mixtures thereof.

In particular, the caloric contribution consisting of heat of dilution plus heat of reaction per kilogram of TDDEF contributed by the additive is between 1.5 and 5.0 kcal.

Also particularly, the contribution of hydroxyl groups (OH) per kilogram of TDDEF provided by the additive is between 5.5 and 8.5 grams of OH.

Essentially, the feed consists of a TDDEF, wherein the pH greater than 9 of the ration with the addition of additive is established with a moisture percentage of 12.5% to 13.5% w/w as a reference.

Also essentially, if the TDDEF has a moisture percentage different from 12.5% to 13.5% w/w, the effective amount of additive to be added to the ration is adjusted taking 12.5% to 13.5% w/w moisture in the ration as a reference value.

### BRIEF DESCRIPTION OF THE FIGURES

FIG. 1 schematically shows the rumen of a ruminant with a pH of approximately 6.8, after consuming grass.

FIG. 2 schematically shows the rumen of a ruminant with a pH lower than 5.8, after consuming a large proportion of carbohydrates and proteins added to the diet.

FIG. 3 schematically shows the rumen of a ruminant with a pH of approximately 6.8, after consuming grass and grains with the additive of the invention.

FIG. 4 shows a graph of the solubility of soy protein isolate as a function of pH (Sarai Serrano Vallecios, M., 2015).

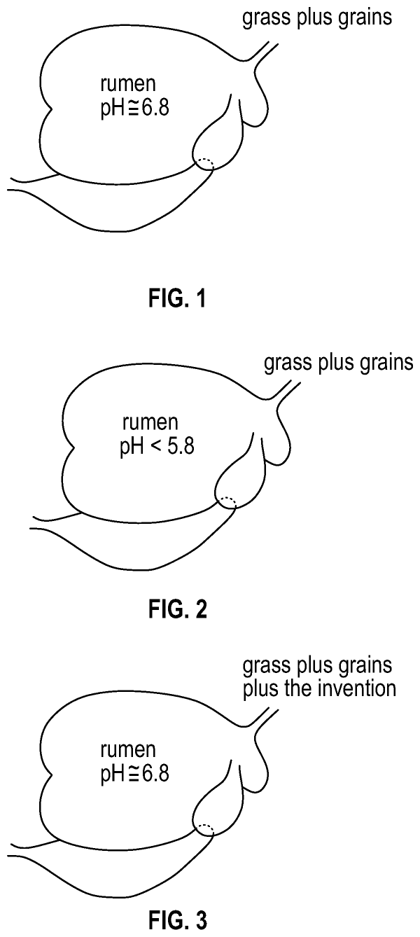

### DETAILED DESCRIPTION OF THE INVENTION

With the aim of overcoming the drawbacks of prior art, the object of the present invention is an additive for ruminant animal nutrition, comprising the following components: sodium hydroxide, calcium oxide, calcium hydroxide and sodium sulfate, such that said components are present in the following proportions expressed in % w/w:

| | | Inclusion of | | |
|---|---|---|---|---|
| | | components in | | |
| | | the invention | | Additive in TDD |
| Solid form component | | % min. | % max. | (in AFB) |
| NaOH | Sodium | 25 | 69 | An effective amount |
| | hydroxide | | | necessary for the pH |
| CaO | Calcium oxide | 0 | 31 | of the TDDEF to be |
| Ca(OH)₂ | Calcium | 0 | 50 | greater than 9 |
| | hydroxide | | | |
| Na₂SO₄ | Sodium | 0 | 19 | |
| | sulfate | | | |

| | |
|---|---|
| An inert excipient that is | Q.S. to reach |
| food-grade acceptable | 100% w/w |

wherein Q.S. stands for quantum satis (sufficient quantity);
wherein TDD stands for “total daily diet”, which is the sum of the different rations that an animal ingests in a day;
wherein TDDEF stands for “total daily diet excluding forage”; and
wherein AFB stands for “as-fed basis” which is the ration including water expressed as moisture.

Particularly, the inert food-grade excipient is selected from the group consisting of shell, silicates, limestone, aluminosilicate, and mixtures thereof. Specifically, the aluminosilicate excipient is selected from the group consisting of zeolite, bentonite, diatomaceous earth, kaolin, and mixtures thereof.

Furthermore, each component of the additive for ruminant animal nutrition is considered as a pure substance in its formulation.

The additive for ruminant animal nutrition of the present invention is capable of providing heat, wherein this heat results from the combination of dilution heat and reaction heat, and consists of an amount ranging from 1.5 to 5.0 kcal per kilogram of TDDEF. In particular, the heat contribution from dilution heat is originated by NaOH and Na₂SO₄, and the heat contribution from reaction heat is originated by CaO and H₂O.

Furthermore, the contribution of hydroxyl groups (OH) per kilogram of TDDEF provided by the additive for ruminant animal nutrition is between 5.5 and 8.5 grams of OH.

Moreover, the contribution of hydroxyl groups (OH) to TDDEF is carried out through any of the following: formulation of the additive, reaction of CaO with H₂O present in TDDEF, during the food preparation process with the additive, ruminal digestive system, and their combinations.

The additive for ruminant animal nutrition is added to a ruminant feed, wherein the feed is a regular diet consisting of feed ingredients for ruminants, wherein the diet is composed of balanced rations consisting of concentrated feeds, forages, minerals, food additives, and mixtures thereof in the form of a totally mixed ration (TMR).

In another embodiment, the present invention also provides a ruminant feed comprising an effective amount of the additive for ruminant animal nutrition, wherein said effective amount is necessary to achieve a pH greater than 9 in TDDEF. Essentially, the feed consists of a regular diet consisting of feed ingredients for ruminants.

According to the document “Balancing Rations ASC-12” by John T. Johns, Roy Burris, Nelson Gay, and David Patterson (1991), a ration is the amount of food that an animal receives in a 24-hour period. A balanced ration is a totally mixed ration (TMR) consisting of an amount of feed that will provide the appropriate quantity and proportions of nutrients necessary for an animal to achieve a specific goal, such as growth, maintenance, lactation, or gestation.

Particularly, the diet consists of balanced portions composed of concentrated feeds, forages, minerals, food additives, and mixtures thereof in the form of TDD or TMR.

In one embodiment, the concentrated feeds are selected from corn, sorghum, barley, wheat, oats, wheat bran, soybean hulls, corn semolina, gluten, malt, dried distillers grains (DDG), glycerol, whey, whey permeate, molasses, beef tallow, blood meal, vegetable fat, animal fat, vegetable protein, animal protein, soybean meal, extruded soybean meal, soybean pellets, cereal meals, and mixtures thereof.

In another embodiment, the forages are selected from hays, straws, pastures, grasslands, cobs, cotton hulls, corn stalks, and mixtures thereof.

In yet another embodiment, the minerals are selected from sodium, potassium, magnesium, calcium, phosphorus, sulfur, manganese, selenium, chromium, cobalt, iodine, iron, zinc, copper, and mixtures thereof.

In still another embodiment, the food additives comprise technological additives selected from adsorbents, binders, anti-caking agents, antioxidants, anti-humectants, preservatives, emulsifiers, stabilizers, thickeners, gelling agents, acidity regulators, humectants, and mixtures thereof; sensory additives selected from colorants, pigments, flavors, palatability enhancers, and mixtures thereof; nutritional additives, vitamins, trace elements, organic micro-minerals, amino acids, peptides, urea, urea derivatives, and mixtures thereof; zootechnical additives selected from enzymes, probiotics, prebiotics, postbiotics, acidifiers, plant extracts, antioxidants, and mixtures thereof; and antimicrobial additives selected from ionophores, antibiotics, hormones, and mixtures thereof.

Essentially, the caloric contribution consisting of dilution heat plus reaction heat per kilogram of TDDEF contributed by the additive is between 1.5 and 5.0 kcal.

Likewise, the contribution of hydroxyl groups (OH) per kilogram of TDDEF contributed by the additive is between 5.5 and 8.5 grams of OH.

Typically, the feed consists of a TDDEF, wherein the pH greater than 9 of the ration with added additive is established with a moisture percentage of 12.5% to 13.5% w/w as a reference.

Specifically, if the TDDEF has a moisture percentage other than 12.5% to 13.5% w/w, the effective amount of additive to be added to the ration is adjusted taking 12.5% to 13.5% w/w moisture in the ration as a reference value.

In order to explain in detail the scope of the present invention, the work of Bergen et al. (1977) was chosen as a model, which describes the productive limits of ruminal fermentation:
i) Fermentation of carbohydrates and generation of adenosine triphosphate (ATP) (Y_{ATP}).
ii) Heat and gas losses during ruminal fermentation, and mitigation of greenhouse gas production.
iii) Protein synthesis of ruminal microorganisms, the role of dilution in bacterial growth rate.
iv) Manipulation of fermentation products and growth efficiency in cell yield.
v) The role of passage rate and substrate degradation in the rumen.
vi) The role of ciliated protozoa in ruminal fermentation and host metabolism.

### i) Fermentation of Ruminal Carbohydrates and Generation of Adenosine Triphosphate (ATP) (Y_{ATP}).

The key question is: How can ruminal fermentation be manipulated in a way that optimal production of final products of nutritional value is accompanied by an optimal response in net microbial cell growth?

The main source of energy (ATP) in the rumen is obtained from the fermentation of carbohydrates such as starch, cellulose, pectins, and hemicellulose into volatile fatty acids (VFAs). The most important factor influencing ruminal microbial fermentation is the composition of the diet. Alteration of passage rate and fermentation type result from the manipulation of dietary carbohydrates and their physical form. However, the nature of the change in terms of microbial population and its energetic consequences is not clearly understood. Knowledge about dilution or passage rate and intermicrobial interactions, especially in terms of Y_{ATP} yields, is incomplete. While changes in ecological niches constantly occur in the rumen, resulting in shifts in microbial population dynamics due to adaptation, Wolin et al. (1975) suggested that fermentation end products could be quite different for specific ruminal microorganisms depending on the substrate carbohydrate effect and passage rate.

Hobson (1965), and Stouthamer and Bettenhaussen (1973) have shown that microorganisms will shift their fermentation to less efficient metabolic pathways as passage rate increases because they have lower maintenance requirements in response to a decrease in growth rate. However, there are cases of microorganisms that do not behave according to this mechanism of action. Wolin et al. (1975) and Thompson et al. (1975) proposed an in vivo increase in dilution rate through the incorporation of a mineral salt mixture in a diet, resulting in an increase in passage rate negatively correlated with the molar proportion of propionate and positively correlated with the molar proportion of acetate, with no difference in total VFA production.

Providing large amounts of fermentable carbohydrates has shown that, although most of them will be fermented in the rumen, a significant portion will escape to the lower intestine and undergo secondary fermentation (Orskov et al., 1971; Karr et al., 1966). Considerable amylolytic, cellulolytic, proteolytic, deaminolytic, and ureolytic activity has been detected in the large intestine, favoring digestion (Orskov et al., 1969, 1970; Hecker, 1971; Mann and Orskov, 1973).

Berger et al. (1979) investigated changes in corn cob fiber digestion using NaOH in solution and observed, similar to Jayasuriya et al. (1975), Rexen et al. (1976), and Levy et al. (1977), that the digestibility of dry matter (DM) increases as the level of sodium hydroxide (NaOH) increases. The solubilization of hemicellulose would explain much of the improvement in DM digestibility with NaOH treatment.

The additive for ruminant animal nutrition of the present invention consists of alkalizing the rumen to improve the availability of energy and protein from grains and protein products for the microbiota, thereby balancing the rumen pH and generating VFA.

### ii) Heat and Gas Losses During Ruminal Fermentation, and Mitigation of Greenhouse Gas Production.

Ruminants have an advantage in their digestive system as they are able to extract energy and protein from substrates that non-ruminants cannot. However, the actual energy losses resulting from ruminal fermentation exceed the energy losses associated with digestion reactions in non-ruminants. The goal is not to eliminate ruminal fermentation in the digestion process; rather, the present invention aims to improve the productive efficiency of the animal by reducing energy losses generated by heat from fermentation and methane production.

The heat of fermentation is an exponential expression of the total microbial energy efficiency (Walker, 1965). It is the free energy that is dissipated as a result of inefficiencies in microbial metabolic activity in the rumen and, by definition, is not associated with energy loss in maintaining the microbial population. The quality and quantity of the food have a marked influence on its variability, affecting the type of fermentation developed, and the selection of diet components is crucial to mitigate the problem (Blaxter, 1962).

The production of methane in the rumen is subject to fewer variables than fermentative heat and it seems that it can be controlled through manipulation of the diet. These variables are available in numerous reviews such as: Bryant, 1965; Stadtman, 1967; Demeyer and Van Nevel, 1975. Meanwhile, Hungate et al. (1966) concluded that methane generally constitutes between 15% and 30% of the total ruminal gases, and its production is more influenced by factors such as the physicochemical nature of the feed and intake level.

Highly digestible feed results in less methane production per unit of consumed caloric energy (Blaxter and Clapperton, 1965). It is established that the main precursors of ruminal methane are H₂ and CO₂ (Hungate et al., 1970).

Ruminants contribute 5.7 gigatons of CO₂ per year, representing approximately 80% of livestock sector emissions, and the CH₄ emitted by ruminants contributes 40% of sector emissions (Asselstine et al., 2021). To reduce greenhouse gas emissions, increasing productivity is the most important factor. Hristov et al. (2013) describe the most determining practices as food quality, use of hormones and growth promoters, genetics, health and mortality reduction, herd fertility, crossbreeding and management strategies, precocity, fecundity, ease of calving, peripartum care, reduction of stress factors, and assisted reproductive technology.

Through the additive for ruminant animal nutrition of the present invention, food consumption was optimized, reducing the food required to produce meat by 13% to 46% through improved conversion (Vittone et al., 2023).

iii) Synthesis of Ruminal Microbial Proteins.

The role of dilution rate or passage rate (D), which is the fraction of ruminal volume displaced per hour, and the maintenance coefficient in the efficiency of cellular growth in ruminal fermentation can be compared to a continuous anaerobic microbial culture. Normally, two classes of microorganisms are distinguished under natural ecological conditions: aerobic organisms that use excess oxygen as an electron acceptor and therefore have a high potential for ATP generation from a substrate, and anaerobic organisms that use a variety of limiting metabolites as electron acceptors and consequently have much lower potential for ATP generation from a given substrate. The limiting factors for microbial growth under aerobic and anaerobic conditions are carbon and energy, respectively (Gunsalus and Shuster, 1961). Hungate et al. (1966) proposed that the availability of energy for microbial growth was the limiting factor and, therefore, there must be an upper limit to microbial cell production in the rumen. Anaerobic microorganisms in the rumen are an important source of protein for the host and provide the only mechanism for the utilization of non-protein nitrogen (NPN). In order to achieve high productivity, the quantitative aspects of microbial cell synthesis are important for animals in production. Hungate (1966) calculated that rumen fermentation can produce around 10 g of microbial protein per 100 g of fermented carbohydrate. This value represents an upper limit of synthetic capacity in anaerobic rumen fermentation. Purser (1970) calculated that this level of protein synthesis was equivalent to 18.3 g of digestible protein per Mcal of digestible energy.

Stouthamer and Bettenhaussen (1973) estimated maintenance coefficients at various growth rates and demonstrated that at slow growth, microorganisms have much higher maintenance, so the Y_{ATP} value is determined by the growth rate, which in turn depends on: 1) the dilution rate and 2) the maintenance coefficient in any physiological situation. Isaaeson et al. (1975) found that at low D, approximately 55% of the energy derived from glucose was used for maintenance, while at high D, only 15% of the available energy was used for maintenance.

Given that there are wide fluctuations in ruminal osmolality from hypotonic to hypertonic, approximately 250 mOsm/L to approximately 400 mOsm/L respectively, after feeding (Bergen, 1972), microbial growth efficiency could be affected, so increasing feeding frequency stabilizes these fluctuations. Attempts to manipulate the passage rate must take into account: (1) a decrease in feed digestibility; (2) a decrease in ruminal protozoa numbers (Coleman, 1975), thus considering the lower amount of protein provided by this source; (3) changes in the ratio between microbial cells; and (4) changes in fermentation end-products (Van Soest, 1975). Finally, as D or microbial growth rate increases, microorganisms will shift their fermentation to less efficient pathways, but this can be compensated for due to lower maintenance requirements (Hobson, 1965). Matin et al. (1976) have shown that increasing dilution rate influences bacterial enzyme activities.

The interaction of volatile fatty acids (VFAs) with urea secretion and ammonia absorption appears to be important evolutionary adaptations of ruminants to regulate the fermentation process (Aschenbach et al., 2011). When ruminants are on low-protein diets in their natural habitats, endogenously secreted nitrogen acts as a pacemaker for microbial fermentation. The positive effect of VFAs on the urea cycle allows animals to utilize available carbohydrates, while excess ammonia is used to buffer and transfer protons out of the rumen. Conversely, the fermentation rate can also be reduced to prevent excessive fermentation whenever nitrogen intake is moderate. When pH drops too low and VFAs accumulate, urea entry decreases (Abdoun et al., 2010), thereby reducing microbial growth and fermentation rate. Unfortunately, this latter regulatory pathway can be expected to be overridden in most high-producing cattle due to excessive protein feeding; nevertheless, the interactions between VFAs, ammonia absorption, and urea availability are of great importance for regulating microbial dynamics and ruminal pH regulation.

The inclusion of the additive for animal nutrition of the invention in ruminant diets increases ruminal pH and enhances protein solubility. Additionally, it increases the production of volatile fatty acids (VFAs) available to associate with NH₃ via microbial metabolism and produce true protein.

In experiments conducted to evaluate the present invention in different categories of ruminants, the additive for ruminant animal nutrition of the present invention was used as components of different balanced feeds to assess animal behavior, feeding habits, meat yield, meat quality, blood parameters, dry matter intake, conversion, and carbon footprint, among other zootechnical parameters. Twenty-two kilograms of the additive for ruminant animal nutrition of the present invention were included per ton of pelleted balanced feed, wherein the additive provided NaOH between 25% and 69% w/w, CaO between 0 and 20% w/w, Ca(OH)₂ between 10% and 50% w/w, and Na₂SO₄ between 6% and 15% w/w for the different categories and species tested, plus the addition of limestone or wheat semita plus limestone in equal parts as excipients to reach 100% in early weaning, rearing, and finishing feeds.

The pelleted balanced feeds from all the tests containing the additive of the invention had a pH between 9.84 and 12.91.

The alkaline pH of the balanced feeds allowed for the gradual adaptation during diet changes from traditional to pelleted feeds, as it caused a sufficient decrease in consumption for the animals to adapt without the need for an intermediate or “adaptation” formula, thus simplifying management and reducing labor hours in meat production.

The AOAC 943.02 technique is recommended for measuring pH in solid diets, while the AOAC 981.12 and AOAC 942.15 techniques are recommended for measuring pH in liquid presentations.

### iv) Manipulation of Fermentation Products and Growth Efficiency in Cellular Yield.

Productive efficiency can be optimized in the rumen through processes and manipulation of microorganisms to achieve more favorable fermentation end products, i.e. volatile fatty acids (VFAs) and microbial cells advantageous for animal production.

Danielson et al. (2017) reported that within the rumen, methanogenic archaebacteria use H₂ to reduce CO₂ for methane (CH₄) production, thus increasing greenhouse gas emissions.

Van Nevel et al. (1969) and Rufener and Wolin (1968) demonstrated that competition for electrons by rumen methanogens diverts fermentation away from inefficient pathways, such as ethanol, lactate, formate, and possibly succinate, towards acetate production, which yields more ATP available for growth. Related to this discussion is the observation that some strains of Selenomonas ruminantium, although they produce only trace amounts of H₂ in pure culture, substantially increase H₂ production in the presence of methanogenic bacteria (Scheifinger et al., 1973), as defined in the previous section (ii), paragraph 4 of the respective text.

On the other hand, in vivo results (Jackson et al., 1971) have found increased microbial cell yield with increased propionate production. However, these researchers altered the physical form of the diet through grinding and pelleting, which may have increased passage or dilution rate and thus the rate of ruminal microbial growth.

Carbohydrate fermentation releases organic acids that easily lower ruminal pH (Allen et al., 1997; Russell and Rychlik, 2001). In contrast, protein or NPN fermentation may release excess ammonia, associated with proton release and pH increases (Wang and Fing, 1996). This is associated with energy loss from the NH₄-urea cycle.

Sartín et al. (2003) conclude that physiological and pathological alterations in food intake are the result of a complex interaction between nutrient molecules, hormones, and nutrients in the gastrointestinal tract, which have specific final actions in the brainstem, and hypothalamic neurons culminate in a feeding response.

By adding the additive for animal nutrition of the invention to ruminant feed, the population of cellulolytic microorganisms is increased at the expense of methanogenic ones, H₂ combines with OH to form H₂O and does not metabolically end up as methane.

### v) the Role of Passage Rate and Substrate Degradation in the Rumen.

The rate of substrate disappearance in the rumen is a combined function of outflow or passage rate and degradation rate, where (1) particle size and (2) specific gravity are the two primary factors controlling the potential exit of a feed particle from the reticulorumen (Balch and Campling, 1965). A poorly digestible forage will be retained longer, grinding of this forage will increase the disappearance rate from the rumen, and therefore, intake. An increase in rumen disappearance rate will actually decrease fiber digestion (Van Soest, 1975). The same phenomenon is not easily observed with more digestible feeds such as grains, as the digestion rate generally exceeds the rumen outflow rate (Hungate, 1966).

A similar treatment to the ruminal disappearance of carbohydrates used by Hungate (1966) is depicted for protein sources. It is clear that an excess of bypass protein as a source of N in ruminant diets will limit microbial growth and VFAs production.

This point was extensively demonstrated in feeding trials in steers by Schmidt et al. (1973) with formaldehyde-treated soybean, bacterial protein production requires optimal levels of NH₃—N and possibly amino acids as well (Maeng and Baldwin, 1976a,b; Maeng et al. (1976) which are necessary for microbial growth. Estimates vary from 5 to 23 mg NH₃—N/100 milliliters of ruminal fluid (Satter and Slyter, 1974; Mehrez and Orskov, 1976).

Protein hydrolysis allows for the formation of small molecular weight peptides, increasing solubility. According to Panyam and Kilara (1996), solubility may also be related to an increase in polypeptide molecules with exposed charges. Solubility characteristics are thermodynamically affected by the balance between protein-protein and protein-solvent interactions (Horax, 2014a). The ionic interaction between the protein and water results in increased protein solubility.

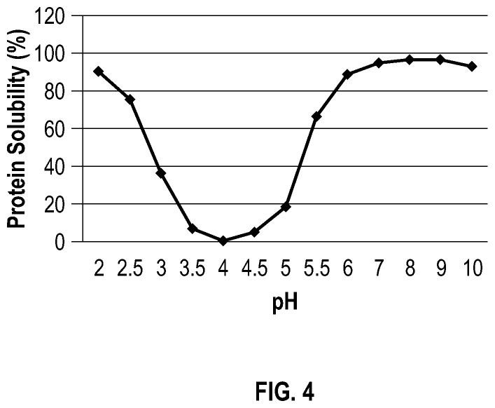

Soy protein has its isoelectric point near pH 4.0, where the protein reaches maximum coalescence and its lowest solubility (Sarai Serrano Vallecios, M., 2015). Due to the pH conditions, protein-water interactions are minimal, causing protein precipitation. The solubility profile of the protein at different pH values is shown in FIG. 4, and it can be observed that non-hydrolyzed soy protein isolate has its highest solubility (>80%) at alkaline pH ranging from 6.0 to 10.0, as well as at pH 2.0. The lowest solubility (<20%) is observed at acidic pH of 3.5 to 5.0.

The inclusion of the additive for animal nutrition of the invention in ruminant feed leads to an increase in pH and therefore proteins will become more soluble.

### vi) the Role of Ciliated Protozoa in Ruminal Fermentation and Host Metabolism.

The extent of contribution of ciliated protozoa to ruminal carbohydrate and nitrogen metabolism has been debated for a long time. Oxford (1955) argued that protozoa provide the host with a greater source of “animal-like” quality protein, as the protein digestibility and lysine content of protozoa exceed those found in ruminal bacteria (Bergen et al., 1968a,b).

Rumen ciliated protozoa engulf bacteria and utilize them as a source of amino acids and other possible growth factors (Coleman, 1975). Large-sized protozoa species can also engulf and use plant proteins directly and utilize urea as a nitrogen source to synthesize protozoal protein (El-Fouly, 1974). However, the work of Nour et al. (1979) quantified different species of protozoa in sheep, goats, cattle, and buffaloes using cottonseed cake and urea in different proportions and found a high negative correlation between the percentage of dietary urea nitrogen and the time required for doubling the protozoal count in vitro.

The overall rate of bacterial decomposition (S. boris) by mixed protozoa in the rumen was estimated at 5.2% per hour (Jarvis, 1968). From a variety of experimental approaches on bacterial and protozoal protein synthesis as a percentage of total microbial protein synthesis, it has been estimated as 80% and 20% (Pilgrim et al., 1970), 70% and 30% (Hungate et al., 1971), and 50% and 50% (Bucholtz and Bergen, 1973), respectively, in studies using forage-based diets.

Variations in protozoa concentration in the rumen are associated with the type of animal, individual variation, rumen volume, feeding frequency, and rumen pH (Pulser and Mor, 1966).

Protozoa are responsible for the dehydrogenation of fats and the digestion of fiber, as they possess enzymes to digest plant fiber, fungi, and rumen bacteria. They are capable of utilizing free nitrogen in the rumen to convert it into high-quality protein and detoxifying harmful substances such as nitrites and nitrates. They likely play a more important role in the nutrition of animals with insufficient daily rations, and while they have been described as one of the main methane-producing microorganisms in the rumen, they may contribute to the reduction of greenhouse gas production when considering their fiber-digesting capabilities compared to undegraded fiber.

### EXAMPLES

### Example 1: Inclusion of the Additive of the Present Invention for Feed Intake Control in Cattle Rations and Assessment of Conversion Efficiency

### Objective

Evaluate the productive performance of fattening steers with different levels of corn inclusion in pelleted feed along with the additive of the present invention.

### Materials

For this trial, three types of pelleted feed with different corn inclusions and a control with a 40% protein concentrate mixture with corn were used. The compositions of the pelleted feeds and the pelleted concentrate used for each group of steers 62, 70, 62/75, and Control, respectively, are provided in Table 1.

TABLE 1
Composition of the feeds used in the trial for each
group of steers and their basic analytical values.

| | | | | Pelleted |
|---|---|---|---|---|
| | Pelleted Feeds | | | Concentrate |
| Component | 62 | 70 | 75 | 40% CP |
| Corn (kg) | 620 | 700 | 750 | — |
| Soybean Pellet 44 CP | 40 | 40 | 40 | 150 |
| (kg) | | | | |
| Sunflower Pellet 32 | 50 | — | — | 230 |
| CP (kg) | | | | |
| Cotton Pellet 34 CP | 80 | 140 | 140 | 500 |
| (kg) | | | | |
| Soybean Hull Pellet | 161.365 | 71.365 | 21.365 | — |
| (kg) | | | | |
| Urea (kg) | 5 | 5 | 5 | 44 |
| Salt (kg) | 3 | 3 | 3 | 20 |
| Limestone (kg) | 16 | 16 | 16 | 29.65 |
| Additive of the | 22 | 22 | 22 | — |
| invention (kg) (1) | | | | |
| Premix VM (kg) (2) | 2.5 | 2.5 | 2.5 | 25 |
| Rumensin (kg) (3) | 0.135 | 0.135 | 0.135 | 1.350 |
| DM (%) | 89.69 | 89.42 | 89.27 | 92.68 |
| CP (% AFB) | 13.18 | 13.15 | 12.95 | 40 |
| Starch (% AFB) | 41.29 | 46.27 | 49.48 | 1.37 |
| EE (% AFB) | 3.03 | 3.14 | 3.18 | 2.43 |
| ADF | 12 | 9.07 | 6.84 | 18.51 |
| pH | 11.54 | 11.97 | 11.73 | 7.37 |

(1) NaOH (95% purity) 69%, CaO (85% purity) 10%, Ca(OH)₂ (75% purity) 10%, Na₂SO₄ (99% purity) 6%, and limestone as excipient in s.q. to reach 100%.
(2) Premix VM contains (dry basis): 0.0028% vitamin A (300,000 IU/g); 0.004% vitamin D3 (70,000 IU/g); 0.1725% vitamin E (550 IU/g); 0.0225% sodium selenite; 0.0016% calcium iodate; 0.00005% cobalt carbonate; 0.042% copper oxychloride; 0.09% zinc sulfate; 0.045% ferrous sulfate; 0.007% manganese sulfate; 0.007% copper sulfate; 0.2431% sulfur flor; 2.5% magnesium oxide, and wheat semita plus limestone in equal parts as excipient in s.q. to reach 100%.
(3) 30 mg/kg of monensin (Rumensin 200, Elanco Animal Health, Indianapolis).
CP: crude protein.
DM: dry matter.
AFB: As-fed basis.
EE: Ether extract.
ADF: acid detergent fiber.

### Methodology

The fattening experiment was conducted in Ecological model feedlot pens, where the rules of intensive meat production system were followed to preserve animal welfare and the environment. The experiment lasted for a total of 118 days. Fifty-six British breed steers weighing 235 kg BW (Body weight) were used. The animals were individually identified with electronic buttons and randomly assigned to four treatments of 14 animals each:
Self-feeding of pelleted feed with the inclusion of the additive and 62% corn in the formula→62

Self-feeding of pelleted feed with the inclusion of the additive and 70% corn in the formula→70.

Self-feeding of pelleted feed with the additive and 62% corn in the formula, and from day 50, offering pelleted feed formulated with 75% corn→62/75
Self-feeding of corn-based grain rations and protein concentrate.

### Control

The three experimental groups 62, 70, and 62/75 were fed pelleted feed with the inclusion of the additive of the invention from the first day of feeding without the need to provide any type of fiber, i.e., without adaptation.

In the Control group, during the adaptation period, a ground mixture formulated with 84% corn grain (50% whole and 50% ground), 10% commercial protein concentrate (40% crude protein), and 6% salt (NaCl) was provided. The animals also had ad libitum access to hay until the 5th week. From the 6th week onwards, a ration formulated with 90% whole corn grain and 10% of the same protein concentrate was offered for self-feeding.

All animals were weighed at 7-day intervals (Hook AT45π electronic scale) during the first month and every 14 days for the rest of the experiment to determine the average daily weight gain (ADG) and total weight gain (TWG). Dry matter intake (kg/head/day and % BW) was estimated by the difference between the supplies of each feed resource and the remaining feed at the end of the experiment (oat roll in corn stubble: 92% DM and 30% waste, balanced feed: 90% DM).

The feed conversion ratio was estimated by the ratio between the total consumption (dry basis) and the sum of the live weight gain of each group.

A total of 49 steers were sent for slaughter as they reached a target weight of 380 to 400 kg. The weight of half carcasses was recorded and the hook yield (8% trimming) was estimated. Additionally, the average duration of the fattening period was determined according to the type of feed.

The statistical analysis was conducted using Infostat 2020 software. ANOVA was applied for the variables of weight evolution, body composition, and beef yield (Tukey's test). The correlation coefficient between variables of interest was also analyzed. A significance level of 5% (α=0.05) was used in all cases.

### Results and Brief Discussion

Table 2 presents the weight evolution variables of fattened steers with different levels of corn inclusion in pelletized feed with the additive of the invention. In all cases, similar values of ADG (kg/head/day; p=0.7564 and % BW; p=0.644) and TKG (p=0.5584) were observed. Therefore, the final weight was also similar among groups (p=0.6830).

TABLE 2
Weight evolution of fattened steers with different levels of corn inclusion
in pelletized feed with the additive of the invention versus control.

| | 62 | 70 | 62/75 | Control |
|---|---|---|---|---|
| Initial weight | 236.7 ± 26.5 | 234.9 ± 33.5 | 238.3 ± 37.4 | 231.4 ± 33.8 |
| Final weight | 380.3 ± 38.5 | 380.6 ± 24.8 | 393.2 ± 43.6 | 390.0 ± 25.6 |
| Fattening duration (days) | 105 ± 18 | 101 ± 18 | 105 ± 15 | 107 ± 19 |
| ADG (kg/head/day) | 1.4 ± 0.4 | 1.4 ± 0.3 | 1.5 ± 0.4 | 1.5 ± 0.2 |
| ADG (% BW) | 0.5 | 0.5 | 0.5 | 0.5 |
| TKG (kg/animal) | 143.6 ± 27.2 | 145.6 ± 38.8 | 154.9 ± 36.4 | 158.6 ± 24.8 |

The peak ADG for the groups with the additive of the invention occurred at 15 days after starting the experiment. Groups 62 and 62/75 showed similar ADG, and in the latter group, when switched to the formula with 75% corn (day 50), the animals gained an additional 0.5 kg/day compared to the 62 group. As the weeks went by, the difference decreased to the point of returning to an inverse relationship of ADG, with a difference of 0.7 to 0.5 kg/head/day in favor of the 62 group.

The 70 group showed the greatest stability over time. At all recording moments, the average ADG was equal to or greater than 1.4 kg/head. Only on day 78, a decrease in ADG was observed in both the 70 and 62 groups.

Table 3 presents the consumption and conversion of the different groups. In the Control group, the animals remained on a ground diet with salt and access to hay for 43 days. During this period, they consumed 5.6 kg DM/head/day of ration and 3.2 kg DM/head/day of hay (total=8.8 kg DM/head/day). This represented a distribution of consumption between the two resources of 64% and 36%, respectively. For the rest of the period, they had an average daily consumption of 9.9 kg DM/head of concentrated ration. The average DM intake/head/day was 9.5 kilograms.

In the 62/75 group, the animals had a consumption of 5.0 kg DM/head/day for the first formula (62) and 7.5 kg DM/head/day for the second (75). Similar to previous experiments, the pelletized feed with the additive of the invention showed improved results in animal efficiency compared to traditional concentrated diets. The animals in the 62/75 treatment had the lowest consumption and the best conversion. It took 13%, 18%, and 46% more feed in the 62, 70, and Control groups, respectively, to produce the same kg of live weight. The improvement in conversion in this study was related to the lower consumption (% BW).

TABLE 3
Consumption and conversion of fattened steers with
different levels of corn inclusion in pelletized
feed with the additive of the invention.

| | 62 | 70 | 62/75 | Control |
|---|---|---|---|---|
| Consumption (kg DM/animal) | 695.6 | 734.9 | 663 | 1,118.4 |
| Estimated daily consumption | 6.6 | 7.3 | 6.3 | 9.5 |
| (kg DM/animal/day) | | | | |
| Estimated consumption (% | 2.1 | 2.4 | 2 | 3.1 |
| BW) | | | | |
| Conversion (kg DM/kg LW) | 4.8 | 5 | 4.3 | 7.1 |

Table 4 presents the average duration of the fattening period for each group, the weight of the animals sent to slaughter, and the beef yield. The ADG and TWG, the results were similar between treatments. The duration of the fattening period was similar among groups (p=0.8641). The same occurred with the weight of the beef (p=0.5896) and the yield (p=0.0803).

TABLE 4
Duration of fattening period and beef yield of fattened steers with different
levels of corn inclusion in pelletized feed with the additive of the invention.

| | 62 | 70 | 62/75 | Control |
|---|---|---|---|---|
| (n) | 10 | 13 | 12 | 14 |
| Fattening period (days) | 105 ± 18 | 101 ± 18 | 105 ± 15 | 107 ± 19 |
| Slaughter weight (kg) | 394.3 ± 34.0 | 383.3 ± 27.5 | 405.3 ± 33.8 | 390.0 ± 25.6 |
| Slaughter weight w/trimming | 362.8 ± 31.3 | 352.6 ± 25.3 | 372.8 ± 31.1 | 358.8 ± 23.5 |
| (kg) | | | | |
| Beef weight (kg) | 210.7 ± 18.4 | 204.7 ± 17.7 | 211.2 ± 14.5 | 202.7 ± 15.4 |
| Beef yield (%) | 58.1 ± 0.0 | 58.0 ± 0.0 | 56.7 ± 0.0 | 56.5 ± 0.0 |

### Conclusions

The percentage of corn inclusion resulted in a similar weight evolution between groups, including the Control. The diet changes (Control and 62/75) caused different degrees of diarrhea in the animals. However, this group presented the best conversion index, even when compared to the other treatments where the balanced diet with the additive of the invention was used. It required 13%, 18%, and 46% more feed in the 62, 70, and Control groups, respectively, to produce the same kg of BW.

### Example 2: Inclusion of the Additive of the Invention in Bovine Weaning Rations for Consumption Control and Improved Conversion

### Objective

To evaluate the productive response of weaned calves fed with a growing balanced diet including the additive of the invention.

### Materials

For this trial, two types of pelleted balanced feeds were used, both at 16% growing, one being traditional and the other with the additive of the invention included in the formula.

TABLE 5
Composition of the feeds used in the trial for each group
of weaned calves and their basic analytical values.

Balanced Feed

| Component | Additive | Control |
|---|---|---|
| Corn (kg) | 469.365 | 469.365 |
| Soybean pellet 44 CP (kg) | 80 | 80 |
| Cotton pellet 34 CP (kg) | 170 | 170 |
| Soybean hull pellet (kg) | 250 | 250 |
| Urea (kg) | 5 | 5 |
| Salt (kg) | — | 6 |
| Limestone (kg) | — | 16 |
| Additive of the Invention (kg) | 22 | — |
| (1) | | |
| Premix VM (kg) (2) | 3.5 | 3.5 |
| Rumensin (kg) (3) | 0.135 | 0.135 |
| DM (%) | 90.07 | 90.38 |
| CP (% AFB) | 16.20 | 16.16 |
| Starch (% AFB) | 34.74 | 34.74 |
| EE (% AFB) | 2.97 | 2.97 |
| ADF | 17.28 | 17.31 |
| pH | 9.84 | 6.43 |

(1) NaOH (95% purity) 27%, CaO (85% purity) 20%, Ca(OH)₂ (75% purity) 30%, Na₂SO₄ (99% purity) 15%, and limestone as an excipient in s.q. to reach 100%.
(2) Premix VM contains (dry basis): 0.0028% vitamin A (300,000 IU/g); 0.004% vitamin D3 (70,000 IU/g); 0.1725% vitamin E (550 IU/g); 0.0225% sodium selenite; 0.0016% calcium iodate; 0.00005% cobalt carbonate; 0.042% copper oxychloride; 0.09% zinc sulfate; 0.045% ferrous sulfate; 0.007% manganese sulfate; 0.007% copper sulfate; 0.2431% sulfur flour; 2.5% magnesium oxide, and wheat semolina plus limestone in equal parts as an excipient in s.q. to reach 100%.
(3) 30 mg/kg of monensin (Rumensin 200, Elanco Animal Health, Indianapolis).
CP: Crude protein.
DM: Dry matter.
AFB: As-fed basis.
EE: Ether extract.
ADF: Acid detergent fiber.

### Methodology

The experiment was conducted in an experimental field and lasted for 63 days. Twenty-four Hereford and Hereford x Angus calves, with an average age of 4.0±0.5 months and a live weight (LW) of 111.4±21.6 kg, were weaned. They were randomly assigned to 2 treatments: 2 replicates/treatment, 6 calves/replicate:
Control+hay: Daily supply of balanced feed for calves with 16% crude protein (CP) and 3.0 Mcal metabolizable energy (ME)+alfalfa hay, concentrate to hay ratio of 80:20.
Additive of the invention: Ad libitum access from day one to balanced feed with the additive for consumption regulation incorporated in the pellet, with 16% CP and 3.0 Mcal ME.

In the control treatment, the weaning protocol was initiated with a feed supply of 1% LW and the levels of the balanced feed were increased until there was leftover feed in the feeders after 24 hours on day 15 of the experiment. The group with the additive received the balanced feed ad libitum from the day of weaning. Sufficient feed for 3-4 days was provided. Each pen (replicate) had access to water troughs with permanent water availability, linear feeders (0.5 m/animal), and an allocation of 50 m²/calf. Samples of the balanced feeds and alfalfa hay were taken. They were then weighed and placed in an oven (60° C., 48 h) to estimate the dry matter (DM) content and consumption. The formulations of the balanced feeds in both treatments were isoproteic and isoenergetic.

In 3 moments of the experiment, i.e. days 22, 27 and 29, the distribution of consumption throughout the day was evaluated in one replicate/treatment. The feed was weighed at 3, 7, 10 and 24 hours after feeding to estimate the consumption during the morning, midday, afternoon, evening, and total/day.

The weight of all animals was recorded at 7-day intervals (using an electronic scale Hook® ST 108) to determine the daily live weight gain (DLWG) and total kilograms gained (TKG). The consumption of dry matter (DM) was estimated based on the difference between the daily feed offered and the daily leftover feed (Control+hay) or the feed provided for 3-4 days (additive-treated feed). Conversion was calculated as the ratio between consumption and TKG (kg DM/kg LW).

The statistical analysis of the different variables was performed using Infostat 2018 software. The weight evolution was estimated using repeated measures over time (RMT). ANOVA was conducted for DLWG and TKG (Tukey's test). A significance level of 5% (α=0.05) was used in all cases.

### Results and Brief Discussion

Table 6 presents the weight evolution of the animals during the evaluation period. The weight evolution was similar between treatments. However, at the end of the experiment, the calves in the additive-treated group weighed 18% more than the control group.

TABLE 6
Weight evolution of weaned calves reared on balanced
feed with the additive of the invention.

| | | Additive- | | |
|---|---|---|---|---|
| | Control + | treated | | |
| | hay | feed | SEM | p-value |
| Initial weight (kg) | 111.3 | 111.5 | 0.7 | 0.8921 |
| Final weight (kg) | 180.1 | 191.8 | 4 | 0.1839 |
| DLWG (kg/calf/day) | 1.15 | 1.3 | 0.06 | 0.1617 |
| DLWG (% LW) | 0.8 | 0.9 | 0 | 0.2929 |
| TKG (kg/animal) | 68.8 | 81.5 | 4.2 | 0.157 |

Table 7 presents the consumption and conversion achieved during the experiment. The calves showed similar daily consumption (kg DM/day), but the consumption as a percentage of LW (%) was lower in the group treated with the additive of the invention. For the evaluation period, the inclusion of the additive resulted in improved conversion, and the animals required 15% less feed to produce each kilogram of LW.

TABLE 7
Productive response of weaned British calves reared
on balanced feed with additive vs. control.

| | | Additive- | | |
|---|---|---|---|---|
| | Control + | treated | | |
| | hay | feed | SEM | p-value |
| Consumption (kg | 4.1 | 4.1 | 0 | >0.9999 |
| DM/animal/day) | | | | |
| Consumption (% LW) | 3.2 a | 3.1 b | 0 | 0.0125 |
| Conversion | 3.6 ± 0.2 a | 3,.1 ± 0.2 b | 0.2 | 0.0149 |
| (kg DM/kg LW) | | | | |

a, b: different letters between columns indicate statistical difference (Tukey's test, α = 0.05).

In line with the distribution of consumption throughout the day, a higher frequency of animals was observed in the feeders in the morning in the Control+hay group, associated with the daily supply of feed. The distribution of consumption throughout the day is of fundamental importance to ensure smaller and more uniform intakes, guaranteeing a continuous supply of nutrients for the ruminal flora. This condition was characteristic of the treatment with the additive. For water consumption, a higher frequency was observed in the Control+hay group during the morning and midday, possibly associated with feed intake and the inclusion of hay in the diet, while animals consuming balanced feed with the additive of the present invention were homogeneous throughout the day. Among the behavior observations recorded in the batches consuming the additive-treated feed, a higher frequency of food consumption in the afternoon and evening can be added; a lower number of dominance events in front of the feeder tray and a higher number of animals in a resting position during the day.

### Conclusions

The inclusion of the additive of the present invention in the balanced feed allowed for early weaning of calves without the need for implementing an adaptation protocol to the new diet. The animals exhibited high levels of consumption from the first week post-weaning, associated with satiety and absence of hunger/fasting. Unlike traditional weaning protocols where the use of hay is employed to “attract” calves to the feeder, the use of the consumption-regulating additive allowed for quick access to the feeder without the need for including fiber.

The animals consuming feed with the additive of the invention showed high weight gains from the first week post-weaning (1.3 kg/day), ending the experiment on average 11.7 kg heavier, although this variable did not show statistical difference. However, feed conversion efficiency showed a positive impact in favor of the additive-treated feed. It required 0.5 kg less of feed to produce each kilogram of live weight, representing a 14% reduction in feed fraction.

In addition to improving conversion, the kilograms of feed required to gain one kilogram of weight, it was not necessary to supply feed daily when the balanced feed contained the additive of the invention in its formulation. This strategy makes work easier when implementing this technique in the field.

### Example 3: Response to the Inclusion of Three Different Formulations of the Additive of the Invention for Consumption Regulation in Cattle Rations

### Introduction

Consumption regulation in cattle can be achieved through chemical or physical means. Additionally, consumption is influenced by factors inherent to the animal, the feed, and the environment.

The use of components such as salt, ionophores, magnesium oxide, and calcium sulfate, among others, has been investigated for decades. Currently, they are used in different livestock production modalities, such as supplementation and complete diets, and with different objectives, for example, promotion, reduction or moderation of consumption. However, the variability in results is directly related to the animal category and the feed resources used.

In recent evaluation experiences of the consumption regulation of the additive of the invention, effective consumption control has been determined, while also maximizing weight gain in light calf and heifer categories. Its inclusion in pelleted balanced feeds for different cattle categories allowed for weaning, rearing, and finishing of animals without the need for implementing adaptation protocols or including long fiber in the diets.

### Objective

The objective of this study is to evaluate different formulations of the additive of the invention.

### Materials

For this trial, four pelleted feeds were used, three of which contained the additive of the invention in three different formulations: ADDI 1, ADDI 2, and ADDI 3, plus a control without the additive: ADDI 4, to assess consumption and conversion.

TABLE 8
Composition of the feeds used in the trial for each
group of steers and their basic analytical values.

| | Pelleted Feeds | | | |
|---|---|---|---|---|
| | ADDI 1 | ADDI 2 | ADDI 3 | ADDI 4 |
| Corn | 641 | 641 | 641 | 641 |
| Sunflower Pellet 32% CP | 100 | 100 | 100 | 100 |
| Cotton Pellet 34% CP | 90 | 90 | 90 | 90 |
| Soy Hull Pellet | 120.365 | 120.365 | 120.365 | 120.365 |
| Urea | 5 | 5 | 5 | 5 |
| Salt | 3 | 3 | 3 | 9 |
| Limestone | 16 | 16 | 16 | 32 |
| Additive of the invention (1) | 22 | | | — |
| Additive of the invention (2) | | 22 | | |
| Additive of the invention (3) | | | 22 | |
| Premix VM (4) | 2.5 | 2.5 | 2.5 | 2.5 |
| Rumensin (5) | 0.135 | 0.135 | 0.135 | 0.135 |
| DM (%) | 89.87 | 89.49 | 89.58 | 90.05 |
| CP (% AFB) | 13.05 | 13.02 | 13.02 | 13.02 |
| Starch (% AFB) | 41.89 | 42.62 | 42.37 | 42.19 |
| EE (% AFB) | 3.03 | 3.02 | 3.08 | 3.00 |
| ADF | 13.1 | 13.17 | 12.95 | 12.73 |
| pH | 11.71 | 11.79 | 12.91 | 7.14 |

(1) NaOH (95% purity) 27%, CaO (85% purity) 20%, Ca(OH)₂ (75% purity) 30%, Na₂SO₄ (99% purity) 15%, and limestone as an excipient in s.q. to reach 100%.
(2) NaOH (95% purity) 69%, CaO (85% purity) 10%, Ca(OH)₂ (75% purity) 10%, Na₂SO₄ (99% purity) 6%, and limestone as an excipient in s.q. to reach 100%.
(3) NaOH (95% purity) 40%, Ca(OH)₂ (75% purity) 50%, and limestone as an excipient in s.q. to reach 100%.
(4) Premix VM contains (on a dry basis): 0.0028% vitamin A (300,000 IU/g); 0.004% vitamin D3 (70,000 IU/g); 0.1725% vitamin E (550 IU/g); 0.0225% sodium selenite; 0.0016% calcium iodate; 0.00005% cobalt carbonate; 0.042% copper oxychloride; 0.09% zinc sulfate; 0.045% ferrous sulfate; 0.007% manganese sulfate; 0.007% copper sulfate; 0.2431% sulfur flour; 2.5% magnesium oxide, and equal parts of wheat middlings and limestone as an excipient in s.q. to reach 100%.
(5) 30 mg/kg of monensin (Rumensin 200, Elanco Animal Health, Indianapolis).
CP: Crude protein.
DM: Dry matter.
AFB: As-fed basis.
EE: Ether extract.
ADF: Acid detergent fiber.

### Methodology

The fattening trial was conducted at a farm implementing the ecological feedlot model and lasted for 47 days. Forty Aberdeen Angus steers weighing 399.0±28.4 kg live weight (LW) with 2 teeth were used. They were identified with electronic buttons and randomly allocated into four groups (10 animals each) according to the additive dose: 1) ADDI 1, 2) ADDI 2, 3) ADDI 3, and 4) ADDI 4.

In all cases, the animals had permanent access to the pelleted feed from the first day in silo feeders. The animals remained in paddocks with an allocation of 200 m²/head and permanent access to water.

All animals were weighed at 7-day intervals (Hook AT45π electronic scale) to estimate the daily weight gain (DWG) and total kilograms gained (TKG). On day 42 of the experiment, due to excessive rainfall, it was not possible to record the weight, so the DWG of the fortnight (weeks 6 and 7) was taken into consideration. Consumption (kg/head/day and % BW) was estimated by the difference between the initial feed offer and the remaining feed at the end of the experiment. Feed conversion ratio was estimated as the ratio between total consumption and the sum of TKG for each group.

An observational study was conducted to determine the consistency of feces and the presence of animals with diarrhea. On two days of each week, 10 feces samples per paddock were scored on a scale of 1 to 5 according to their consistency: 1=liquid, 2=soft, 3=optimal, 4=firm, and 5=hard (Bavera & Peñafort, 2006). In addition, the cleanliness of the animals in the tail area was observed: 0=clean, 1=fecal matter adhered around the base of the tail, and 2=presence of fecal matter around the base of the tail and hindquarters (De Paula Vieira et al., 2012).

At the beginning and end of the experiment, the dorsal fat thickness (DFT) and ribeye area (REA) at the 12^{th} left intercostal space were measured. Measurements were taken using a real-time ultrasound machine FALCOVET 100 (Pie Medical, Netherlands) with a 3.5 MHz linear transducer and 20 cm in length, using vegetable oil as a coupling agent.

A group of 36 animals was transported to an export slaughterhouse. The final field weight was recorded, one at the truck during loading (16 h post-field weight) and another upon arrival at the slaughterhouse (21 h post-final weight). By recording the order of entry, the hock number, and the dressing report, the hot carcass weight was determined and the dressing percentage (9% dressing loss, based on final field weight) of each animal was estimated.

Carcass grading was performed, and the percentage of animals with different grades was estimated according to the treatment. Additionally, characteristics of the carcasses, livers, and rumens were observed.

Statistical analysis of the different variables was performed using Infostat 2018 software. The weight evolution was estimated using repeated measures over time (RMT). ANOVA was performed for DWG, TKG, DFT, and REA (Tukey's test). The results of the observational study on feces consistency and animal hygiene were transformed into frequencies and compared using the Kruskal-Wallis test. In all cases, a significance level of 5% (α=0.05) was used.

### Results and Brief Discussion

Table 9 presents the weight evolution of steers fattened with different formulations of the additive of the invention in a balanced feed.

TABLE 9
Weight evolution of steers fattened with different doses
of the additive of the invention in a balanced feed.

| | | | | | p- |
|---|---|---|---|---|---|
| Dose | ADDI 1 | ADDI 2 | ADDI 3 | ADD 4 | value |
| Initial weight | 398.5 ± 28.4 | 398.9 ± 27.1 | 399.3 ± 34.4 | 399.2 ± 28.0 | 0.9999 |
| (kg) | | | | | |
| Final weight (kg) | 515.8 ± 37.5 | 518.5 ± 41.2 | 506.7 ± 37.6 | 479.0 ± 43.0 | 0.1247 |
| DWG | 2.1 ± 0.6 a | 2.1 ± 0.5 a | 2.0 ± 0.4 ab | 1,.5 ± 0.4 b | 0.0198 |
| (kg/head/day) | | | | | |
| DWG (% BW) | 0.5 ± 0.0 a | 0.5 ± 0.0 a | 0.4 ± 0.0 ab | 0.3 ± 0.0 b | 0.0252 |
| TKG (kg/animal) | 117.3 ± 34.7 a | 119.6 ± 33.1 a | 107.4 ± 23.2 ab | 79.8 ± 23.3 b | 0.0156 |

a, b: different letters between columns indicate statistical difference (Tukey, α = 0.05).

No statistical difference was observed in the final weight of the experiment. This may be due to variability within each group. The lowest weight recorded for groups ADDI 1, ADDI 2, ADDI 3 and ADDI 4 was 459 kg, 451 kg, 443 kg and 419 kg, respectively, while the highest weight was 564 kg, 586 kg, 556 kg, and 533 kg. However, a lower weight gain was observed in group ADDI 4 compared to ADDI 1 and ADDI 2 for the variables under study (DWG and TKG).

Table 10 presents the consumption and conversion of steers fattened with different formulations of the additive of the invention in a balanced feed. Groups ADDI 2 and ADDI 1 showed higher consumption than the other groups, as reflected in both kg/head and % BW. The lower consumption and higher DWG obtained in group ADDI 2 led to better conversion efficiency.

TABLE 10
Consumption and conversion of steers fattened with different
doses of the additive of the invention in a balanced feed.

| | ADDI 1 | ADDI 2 | ADDI 3 | ADDI 4 |
|---|---|---|---|---|
| Consumption (kg/head/day) | 11.5 | 9.8 | 11.9 | 8.8 |
| Consumption (% BW) | 2.5 | 2.1 | 2.6 | 2 |
| Conversion (kg DM/kg BW) | 4.6 | 3.9 | 5.2 | 5.2 |

Table 11 presents the results of body composition (DFT and REA) of steers fattened with different formulations of the additive of the invention in a balanced feed. The different doses of the additive did not affect fat deposition or REA growth.

TABLE 11
DFT (mm) and REA (cm²) of steers fattened with different doses
of the additive of the invention in a balanced feed.

| | ADDI 1 | ADDI 2 | ADDI 3 | ADDI 4 | p-value |
|---|---|---|---|---|---|
| Initial DFT (mm) | 7.3 ± 2.1 | 7.5 ± 1.8 | 7.6 ± 1.4 | 7.4 ± 2.3 | 0.987 |
| Final DFT (mm) | 10.9 ± 1.4 | 11.0 ± 1.5 | 12.0 ± 2.2 | 11.2 ± 2.6 | 0.6042 |
| Total DFT increase (mm) | 3.6 ± 1.2 | 3.5 ± 1.2 | 4.4 ± 1.2 | 3.8 ± 1.6 | 0.4576 |
| Initial REA (cm²) | 65.7 ± 7.7 | 59.9 ± 8.0 | 62.7 ± 9.6 | 63.9 ± 8.2 | 0.4865 |
| Final REA (cm²) | 80.7 ± 7.0 | 80.0 ± 7.5 | 78.2 ± 7.6 | 79.5 ± 10.1 | 0.9158 |
| Total REA increase (cm²) | 15.0 ± 9.3 | 20.1 ± 8.7 | 15.6 ± 10.3 | 15.7 ± 9.7 | 0.6155 |

At the end of the fattening period, the 36 heaviest steers were selected and sent for slaughter. Table 12 presents the final weight, carcass weight, and carcass yield. No statistical differences were found in the weight with trimming (9%) or in the carcass weight or yield.

TABLE 12
Final weight, carcass weight, and carcass yield of steers fattened with different
formulations of the additive of the invention in a balanced feed.

| | ADDI 1 | ADDI 2 | ADDI 3 | ADDI 4 | p-value |
|---|---|---|---|---|---|
| (n) | 9 | 10 | 10 | 7 | |
| Final weight (kg) | 522.1 ± 33.4 | 518.5 ± 32.0 | 506.7 ± 32.1 | 497.9 ± 33.2 | 0.5483 |
| Trimmed weight | 475.1 ± 30.7 | 471.8 ± 37.5 | 461.1 ± 34.2 | 453.1 ± 32.8 | 0.5483 |
| (9%; kg) | | | | | |
| Carcass weight | 240.2 ± 20.4 | 248.0 ± 14.6 | 248.4 ± 16.7 | 246.1 ± 17.0 | 0.7223 |
| (kg) | | | | | |
| Carcass yield | 50.7 ± 0.0 | 52.9 ± 0.1 | 54.1 ± 0.0 | 54.8 ± 0.1 | 0.4825 |
| (%) | | | | | |

In terms of productivity, even though the animals in the ADDI 1, ADDI 2, and ADDI 3 groups were heavier (weighed between 10 and 25 kg more) at the end of the experiment, the average carcass weight was similar or even lower than the ADDI 4 group.

### Conclusions

The different formulations of the additive of the invention resulted in differences in the productive performance of the animals. Both formulations of the invention, ADDI 1 and ADDI 2, significantly improved conversion compared to the non-additive diet.

### FINAL CONCLUSIONS

Unexpectedly, it was observed that the additive of the invention can be used in all categories of ruminants. It simplifies tasks and reduces working hours for involved operators. It also modifies the geography of suitable wintering (fattening) places as it allows us to feed without the use of fiber. At the same time, it improves conversion in all categories, and as a result, it reduces greenhouse gas emissions.

The present invention consists of a solid and/or liquid additive, intended for all categories of ruminants, to be incorporated along with other ingredients that constitute compound feed, forages, minerals, food additives, and mixtures thereof in the form of TDD or RMT.

The inclusion of the additive for animal nutrition of the present invention in the feed of ruminants within the scope of the different compositions will depend on the degree of purity or title of the constituent components and the composition of the diet. Likewise, in a TDD constituted by the sum of the different rations that an animal ingests in a day, without including forage such as silage, hay, etc., the pH must be above 9, with the following raw materials being used: sodium hydroxide, calcium oxide, calcium hydroxide, and sodium sulfate.

Therefore, the use of the additive for ruminant animal nutrition in a diet, without considering the addition of forage, with a pH greater than 9.0 is included within the scope of the present invention.

### BIBLIOGRAPHY

1—Dunlop, R. H., and P. B. Hammond.; 1965. D-lactic acidosis of ruminants. Ann. NY Acad. Sci. 119:1109-1132.
2—Lean, I. J.; Golder, H. M.; 2019. Ruminal Acidosis: Beyond pH and Rumen Scibus Camden, NSW, Australia.
3—Owens, F. N., Secrist, D. S. Hill, W. J. and Gill, D. R.; 1998. Acidosis in Cattle: A Review. J. Anim. Sci. 76:275-286.
4—Kleen J. L., Hooijer G. A., Rehage J., and Noordhuizen P. T. M.; 2003. Subacute ruminal acidosis (SARA): a Review.
5—Nagaraja, T. G. and E. C. Titgemeyer; 2007. Ruminal acidosis in beef cattle: The current microbiological and nutritional Outlook. J. Dairy Science 90: E17-E38.
6—Lean, I. J. and Golder, H. M.; 2019. Ruminal Acidosis: Beyond pH and Rumen. Conference: Cornell Nutrition.
7—Li S. E., Yoom I, Scott E. Khafipour E. and Plaizier J. C.; 2016. Impact of Saccharomyces cerevisiae fermentation product and subacute ruminal acidosis on production, inflammation and endotoxins in the rumen and hindgut of dairy cows. J. Dairy Sci. 95:294-303.
8—Bach A., Guasch I., Elcoso G., Chaucheyras-Durant F., Castex M., Fábregas F., García Fruitos E. and Aris A.; 2018. Changes in gene expression in the rumen and colon epithelia during the dry period through lactation of dairy cows and effects of live yeast supplementation. J. Dairy Sci. 101:2631-2640.
9—Tripathi M. K., Santra A., Chaturvedi O. H. and Karim S. A.; 2004. Effect of sodium bicarbonate supplementation on ruminal fluid pH, feed intake, nutrient utilization and growth of lambs fed high concentrate diets. Animal Feed Sci, and Tech. 111:27-39.
10-10—Sarai Serrano Vallecios, M., 2015. Solubility of acid-treated alkalase-isolated soy protein and its functional properties, Special graduation project, Panamerican Agricultural School, Zamorano, Honduras.
11—Sherasia, P. L., Phondba B. T., Hossain S. A., Patel, B. P. and Garg, M. R., 2016. Impact of feeding balanced rations on milk production, methane emission, metabolites and feed conversion efficiency in lactating cows, Indian J. Anim. Res., 50 (4) 2016:505-511. Print ISSN: 0367-6722/Online ISSN: 0976-0555, DOI: 10.18805/ijar.8595.
12—Moss, R. J., Hannah, I. J. C., Kenman, S. J., Buchanan, I. K., Martin, P. R. Editor(s): Stone, G. M., 2000. Response by dairy cows grazing tropical grass pasture to barley or sorghum grain based concentrates and lucerne hay. Asian-Aust. J. Anim. Sci., 13 (Suppl.): 163-168.
13—Johns J. T., Burris R., Gay N., Patterson D.; 1991. Balacing Rations ASC-12. University of Kentucky College of Agriculture. htps://www2.ca.uky.edu/agcomm/pubs/asc/asc12/asc12.htm
14—Bergen, W. G., Yokoyama, M. T.; 1977. Productive limits to rumen fermentation, J. of Animal Science, 46:573.
15—Wolin, M. J.; 1975. Interactions between the bacterial species of the rumen. I. W. McDonald and A. C. I. Warner (Ed.) Digestion and Metabolism in the Ruminant. p. 134. Univ. of New England Publishing Unit, N.S.W. Armidale, N.S.W., Australia.
16—Hobson, P. N.; 1965. Continuous culture of some anaerobes and facultative anaerobic tureen bacteria. J. Gen. Microbiol. 38:167.
17—Stouthamer, A. H. and C. Bettenhaussen; 1973. Utilization of energy for growth and maintenance in continuous and batch cultures of microorganisms. Biochim, Biophys. Acta. 301:53-70.
18—Wolin, M. J.; 1975. Interactions between the bacterial species of the rumen. I. W. McDonald and A. C. I. Warner (Ed.) Digestion and Metabolism in the Ruminant. p. 134. Univ. of New England Publishing Unit, N.S.W. Armidale, N.S.W., Australia.
19—Thompson, D. J., D. E. Beever, D. C. Mundell, M. L. Elderfield and D. G. Harrison; 1975. The effect of altering dilution rate on the pattern of fermentation in the rumen. Proc. Nutr. Soc. 34:111A.
20—Orskov, E. R., C. Fraser and I. McDonald; 1971. Digestion of concentrates in sheep. 1. The effect of increasing the concentration of soyabean meal in a barley diet on apparent disappearance of feed constituents along the digestive tract. Brit. J. Nutr. 25:225.
21—Karr, M. R., C. O. Little and G. E. Mitchell Jr.; 1966. Starch disappearance from different segments of the digestive tract of steers. J. Anim. Sci. 25:652.
22—Orskov, E. R., C. Fraser and R. N. B. Kay; 1969. Dietary factors influencing the digestion of starch in the rumen and small and large intestine of early weaned lambs. Brit. J. Nutr. 23:217.
23—Orskov, E. R., C. Fraser, V. C. Mason and S. O. Mann; 1970. Influence of starch digestion in large intestine of sheep on caecal fermentation, caecal microflora and feacal nitrogen excretion. Brit. J. Nutr. 24:671.
24—Hecker, J. F.; 1971. Metabolism of nitrogenous compounds in the large intestine of sheep. Brit. J. Nutr. 25:85.
25—Mann, S. O. and E. R. Orskov; 1973. The effect of rumen and post-tureen feeding of carbohydrates on the caecal microflora of sheep. J. Appl. Bacteriol. 36:475.
26—Berger L., Klopfenstein T. and Britton R.; 1979. Effect of sodium hidroxide on efficiency of rumen digestión. Journal of An. Sci. 49:1317-1323.
27—Jayasuriya, M. C. N. and Emyr Owen; 1975. Sodium hydroxide treatment of barley straw: Effect of volume and concentration of solution on digestibility and intake by sheep. Anita. Prod. 21:313.
28—Rexen, F., P. Stigsen and F. V. Kristensen; 1977. The effect of a new alkali technique on the nutritive value of straw. Proc. IXth Nutr. Conf. Feed, Univ. Nottingham.
29—Levy, D., Z. Holzer, H. Neumark and Y. Folman; 1977. Chemical processing of wheat straw and cotton by-products for fattening cattle. 1. Performance of animals receiving the wet material shortly after treatment. Anim. Prod. 25:27.
30—Walker, D. J.; 1965. Energy metabolism and rumen microorganisms. In R. W. Dougherty (Ed.) Physiology of Digestion in the Ruminant. p. 296. Butterworths, Inc., Washington, DC.
31—Blaxter, K. L.; 1962. The Energy Metabolism of Ruminants. p. 185. Hutchinson and Co., London, England.
32—Bryant, M. P.; 1965. Rumen methanogenic bacteria. In R. W. Dougherty et al. (Ed.) Physiology of Digestion in the Ruminant. p. 411. Williams and Williams Co., Baltimore, MD
33—Stadtman, T. C.; 1967. Methane fermentation. Annual Rev. of Microbiol. 21:121.
34—Demeyer, D. I. and C. J. Van Nevel; 1975. Methanogenesis, an integrated part of carbohydrate fermentation, and its control. In I. W. McDonald and A. C. I. Warner (Ed.). Digestion and metabolism in the ruminant, p. 366. Univ. New England Publishing Unit, Armidale, N.S.W., Australia.
35—Hungate, R. E.; 1966. The Rumen and its Microbes. Academic Press, NY.
36—Blaxter, K. L. and J. L. Clapperton; 1965. Prediction of the amount of methane produced by ruminants. Brit. J. Nutr. 19:511.
37—Hungate, R. E., W. Smith, T. Bauchop, I. Yu and J. C. Rabinowitz; 1970. Formate as an intermediate in the bovine rumen fermentation. J. Bacteriol. 102:389.
38—Asselstine, V., Lam, S., Miglior, F., Brito, L., Sweett, H., Guan, L., Waters, S. M., Plastow, G. and Canovas, A.; 2021. The potential for mitigation of methane emissions in ruminants through the application of metagenomics, metabolomics, and other-OMICS technologies Journal of Animal Science, 2021, Vol. 99, No. 10, 1-16.
39—Hristov, A. N., Ott, T., Tricarico, J. Rotz, A. Waghorn, G. Adesogan, A. Dijkstra, J. Montes, A. Kebreab, E., Oosting, S. J., Gerber, P. J., Henderson, B., Makkar, H. P. S. and Firkins J. L.; 2013. Mitigation of methane and nitrous oxide emissions from animal operations: III. A review of animal management mitigation options J. Anim. Sci. 2013.91:5095-5113.
40—Vittone, J. S. and Munilla M. E.; 2023. Productive performance of steers with different levels of corn in pelleted balanced feed with RZeta. Technical report INTA EEA Concepción del Uruguay.
41—Gunsalus, I. C. and C. W. Shuster; 1961. Energy yielding metabolism in bacteria. In I. C. Gunsalus and R. Y. Stanier (Ed.) The Bacteria, Vol. II, p. 1. Academic Press, New York.
42—Hungate, R. E.; 1966. The Rumen and its Microbes. Academic Press, NY.
43—Purser, D. B.; 1970. Nitrogen metabolism in the rumen; microorganisms as a source of protein for the ruminant animal. J. Anim. Sci. 30:988.
44—Stouthamer, A. H.; 1969. Determination and significance of growth yields. J. R. Norris and D. W. Ribboxs (Ed.). Methods in Microbiology, Vol. I, Academic Press, New York.
45—Isaacson, H. R., F. C. Hinds, M. P. Bryant and F. N. Owens; 1975. Efficiency of energy utilization by mixed rumen bacteria in continuous culture. J. Dairy Sci. 58:1645.
46—Bergen, W. G., 1972. Role of osmolality in feed intake control in sheep. J. Anita. Sci. 34:1054.
47—Coleman, G. S.; 1975. The interrelationship between rumen ciliate protozoa and bacteria. In I. W. McDonald and A. C. I. Warner (Ed.) Digestion and Metabolism in the Ruminant. p. 149. Univ. of New England Publishing Unit, Armidale, N.S.W., Australia.
48—Van Soest, P. J.; 1975. Physio-chemical aspects of fiber digestion. In I. W. McDonald and A. C. I. Warner (Ed.) Digestion and Metabolism in the Ruminant. p. 351. Univ. of New England Publishing Unit, Armidale, N.S.W., Australia.
49—Hobson, P. N.; 1965. Continuous culture of some anaerobes and facultative anaerobic tureen bacteria. J. Gen. Microbiol. 38:167.
50—Matin, A., Grootjans, A., and H. Hogenhuis; 1976. Influence of dilution rate on enzymes of intermediary metabolism in two fresh water bacteria grown in continuous culture. J. Gen. Microbiol. 94:323.
51—Aschenbach J. R., Peenr G. B., Stumpff F. and Gabel G.; 2011. Ruminal nutrition symposium: Role of fermentation acid absorption in the regulation of ruminal pH. J. Anim. Aci. 89:1092-1107.
52—Abdoun, K., F. Stumpff, I. Rabbani, and H. Martens; 2010. Modulation of urea transport across sheep rumen epithelium in vitro by SCFA and CO2. Am. J. Physiol. Gastrointest. Liver Physiol. 298: G190-G202.
53—Danielsson R., Dicksved, J., Sun, L., Gonda, H., Muller, B., Achnurer, A. and Bertilsson, J.; 2017. Methane production in dairy cows correlates with tumen methanogenic and bacterial community structure. Frontiers in Microbiology vol. 8, Art. 226.
54—Van Nevel, C. J., H. K. Henderickx, D. I. Demeyer and J. Martin; 1969. Effect of chloral hydrate on methane and propionic acid in the rumen. Appl. Microbiol. 17:695.
55—Rufener, W. H. Jr, and M. J. Wolin; 1968. Effect of CCl₄ on CH₄ and volatile acid production in continuous cultures of rumen organisms and in a sheep rumen. Appl. Microbiol. 16:1955.
56—Scheifinger, C. C., B. Linehan and M. J. Wolin; 1973. H₂ production by Selenomonas ruminantium in the absence and presence of methanogenic bacteria. Appl. Microbiol. 29:480.
57—Jackson, P., J. A. F. Rook and K. G. Towers; 1971. Influence of the physical form of a barley grain and barley straw diet on nitrogen metabolism in sheep. J. Dairy Res. 38:33.
58—Allen, M. S.; 1997. Relationship between fermentation acid production in the rumen and the requirement for physically effective fiber. J. Dairy Sci. 80:1447-1462.
59—Russell, J. B., and J. L. Rychlik; 2001. Factors that alter rumen microbial ecology. Science 292:1119-1122.
60—Wang, J., and D. Y. Fung; 1996. Alkaline-fermented foods: A review with emphasis on pidan fermentation. Crit. Rev. Microbiol. 22:101-138.
61—Sartin, J. L., Whitlock, B. K. and Daniel, J. A.; 2003. Triennial Growth Symposium: Neural regulation of beef intake: Modification by hormones, fasting and disease.
62—Balch, C. C. and R. C. Campling; 1965. Rate of passage of digesta through the ruminant stomach. In R. W. Dougherty (Ed.) Physiology of digestion in the ruminant, p. 108. Butterworths, Inc., Washington, DC.
63—Van Soest, P. J.; 1975. Physio-chemical aspects of fiber digestion. In I. W. McDonald and A. C. I. Warner (Ed.) Digestion and Metabolism in the Ruminant. p. 351. Univ. of New England Publishing Unit, Armidale, N.S.W., Australia.
64—Schmidt, S. P., N. A. Jorgensen, N. J. Benevenga and V. H. Brungardt; 1973. Comparation of soybean meal, formaldehyde treated soybean meal, urea and starea for steers. J. Anim. Sci. 37:1233.
65—Maeng, W. J. and R. L. Baldwin; 1976a. Factors influencing rumen microbial growth rates and yields: Effect of urea and amino acids over time. J. Dairy gci. 59:643.
66—Maeng, W. J. and R. L. Baldwin; 1976b. Factors influencing rumen microbial growth rates and yields: Effect of amino acid additions to a purified diet with nitrogen from urea. J. Dairy Sci. 59:748.
67—Maeng, W. J., C. J. Van Nevel, R. L. Baldwin and J. G. Morris; 1976. Rumen microbial growth rates and yields: Effect of amino acids and protein. J. Dairy Sci. 59:68.
68—Satter, L. D. and L. L. Slyter; 1974. Effect of ammonia concentration on rumen microbial protein production in vitro. Brit. J. Nutr. 32:199.
69—Mehrez, H. A. and E. R. Orskov; 1976. Rates of rumen fermentation in relation to ammonia concentration. Proc. Nutr. Soc. 35: 40A.
70—Oxford, A. E.; 1955. The rumen ciliate protozoa: Their chemical composition, metabolism, requirements for maintenance and culture, and physiological importance for the host. Experimental Parasitology. 4:569.
71—Bergen, W. G., D. B. Purser and J. K. Cline; 1968a. Determination of limiting amino acids of rumen isolated microbial proteins fed to rats. J. Dairy Sci. 51:1698.
72—Bergen, W. G., D. B. Purser and J. K. Cline; 1968b. Effect of ration on the nutritive quality of rumen microbial protein. J./Maim. Sci. 27:1497.
73—Coleman, G. S.; 1975. The interrelationship between rumen ciliate protozoa and bacteria. In I. W. McDonald and A. C. I. Warner (Ed.) Digestion and Metabolism in the Ruminant. p. 149. Univ. of New England Publishing Unit, Armidale, N.S.W., Australia.
74—El-Fouly, H. A.; 1974. The use of the marker techniques in nutritional studies in the rumen. Ph.D. Thesis. Cairo Univ.
75—Nour A. M., Abou Akkada A. R., El-Shazly K. and Naga M. A.; 1979. Effect of Increased levels of Urea in the diet on ruminal protozoal counts in four ruminant species. J. of Animal Sci. 49:1300-1304.
76—Jarvis, B. D.; 1968. Types of viable rumen bacteria in bovine rumen fluid. Appl. Microbiol. 16:714.
77—Pilgrim, A. F., F. V. Gray, R. A. Weller and C. B. Belling; 1970. Synthesis of microbial protein from ammonia in the sheep's rumen and the proportion of dietary nitrogen converted into microbial nitrogen. Brit. J. Nutr. 24:589.
78—Hungate, R. E., J. Reidel and R. Prins; 1971. Parameters of rumen fermentation in a continuousiy fed sheep: evidence of a microbial rumination pool. Appl. Microbiol. 22:1104.
79—Bucholtz, H. F. and W. G. Bergen; 1973. Microbial phospholipid synthesis as a marker for microbial protein synthesis in the rumen. Appl. Microbiol. 25:504.
80—Purser, D. B. and R. J. Mour; 1966. Dietary effects of concentrations of protozoa in the rumen. J. Anim. Sci. 25:668.
81—Bavera, G., & Peñafort, C.; 2006. Reading Bovine Feces and its Relationship with Feeding. Produccion-Animal.Com.Ar, 1-9. http://produccion-animal.com.ar/informacion_tecnica/manejo_del_alimento/61-heces_del_bovino_y_relacion_con_la_alimentacion.pdf
82—De Paula Vieira, A., von Keyserlingk, M. A. G., & Weary, D. M.; 2012. Presence of an older weaned companion influences feeding behavior and improves performance of dairy calves before and after weaning from milk. Journal of Dairy Science, 95 (6), 3218-3224. https://doi.org/10.3168/jds.2011-4821

1. An additive for ruminant animal nutrition, comprising the following components: sodium hydroxide, calcium oxide, calcium hydroxide and sodium sulfate, in such a way that said components are present in the following proportions expressed in % w/w:

| | | Inclusion of | | |
|---|---|---|---|---|
| | | components in | | |
| | | the invention | | Additive in TDD |
| Solid form component | | % min. | % max. | (in AFB) |
| NaOH | Sodium | 25 | 69 | An effective amount |
| | hydroxide | | | necessary to achieve a |
| CaO | Calcium oxide | 0 | 31 | pH greater than 9 in |
| Ca(OH)₂ | Calcium | 0 | 50 | TDDEF |
| | hydroxide | | | |
| Na₂SO₄ | Sodium sulfate | 0 | 19 | |
| | |
|---|---|
| Inert excipient that is food- | Q.S. to reach |
| grade acceptable | 100% w/w. |

wherein Q.S. stands for quantum satis (sufficient quantity);
wherein TDD stands for “total daily diet”, which is the sum of the different rations that an animal ingests in a day;
wherein TDDEF stands for “total daily diet excluding forage”; and
wherein AFB stands for “as-fed basis” which is the ration including water expressed as moisture.

2. The additive of claim 1, wherein the inert food-grade acceptable excipient is selected from the group consisting of shell, silicates, limestone, aluminosilicate, and mixtures thereof.

3. The additive of claim 2, wherein the aluminosilicate excipient is selected from the group consisting of zeolite, bentonite, diatomaceous earth, kaolin, and mixtures thereof.

4. The additive of claim 1, wherein each component of the additive for ruminant animal nutrition is considered as a pure substance in its formulation.

5. The additive of claim 1, wherein its capacity of providing heat, which results from the combination of dilution heat and reaction heat, consists of an amount ranging from 1.5 to 5.0 kcal per kilogram of TDDEF.

6. The additive of claim 5, wherein the heat contribution from dilution heat is originated by NaOH and Na₂SO₄.

7. The additive of claim 5, wherein the heat contribution from reaction heat is originated by CaO and H₂O.

8. The additive of claim 1, wherein the contribution of hydroxyl groups (OH) per kilogram of TDDEF provided by the additive for ruminant animal nutrition is between 5.5 and 8.5 grams of OH.

9. The additive of claim 8, wherein the contribution of hydroxyl groups (OH) to TDDEF is carried out through any of the following: formulation of the additive, reaction of CaO with H₂O present in TDDEF, during the food preparation process with the additive, ruminal digestive system, and their combinations.

10. The additive of claim 1, wherein the feed is a regular diet consisting of feed ingredients for ruminants.

11. The additive of claim 10, wherein the diet is composed of balanced rations consisting of concentrated feeds, forages, minerals, food additives, and mixtures thereof in the form of a TDD or totally mixed ration (TMR).

12. A ruminant feed comprising an effective amount of the additive for ruminant animal nutrition of claim 1, wherein said effective amount is necessary to achieve a pH greater than 9 in TDDEF.

13. The feed of claim 12, wherein the feed consists of a regular diet consisting of feed ingredients for ruminants.

14. The feed of claim 13, wherein the diet is composed of balanced rations consisting of concentrated feeds, forages, minerals, food additives, and mixtures thereof in the form of a TDD or totally mixed ration (TMR).

15. The feed of claim 14, wherein the concentrated feeds are selected from corn, sorghum, barley, wheat, oats, wheat bran, soybean hulls, corn semolina, gluten, malt, dried distillers grains (DDG), glycerol, whey, whey permeate, molasses, beef tallow, blood meal, vegetable fat, animal fat, vegetable protein, animal protein, soybean meal, extruded soybean meal, soybean pellets, cereal meals, and mixtures thereof.

16. The feed of claim 14, wherein the forages are selected from hays, straws, pastures, grasslands, cobs, cotton hulls, corn stalks, and mixtures thereof.

17. The feed of claim 14, wherein the minerals are selected from sodium, potassium, magnesium, calcium, phosphorus, sulfur, manganese, selenium, chromium, cobalt, iodine, iron, zinc, copper, and mixtures thereof.

18. The feed of claim 14, wherein the food additives comprise technological additives selected from adsorbents, binders, anti-caking agents, antioxidants, anti-humectants, preservatives, emulsifiers, stabilizers, thickeners, gelling agents, acidity regulators, humectants, and mixtures thereof; sensory additives selected from colorants, pigments, flavors, palatability enhancers, and mixtures thereof; nutritional additives, vitamins, trace elements, organic micro-minerals, amino acids, peptides, urea, urea derivatives, and mixtures thereof; zootechnical additives selected from enzymes, probiotics, prebiotics, postbiotics, acidifiers, plant extracts, antioxidants, and mixtures thereof; and antimicrobial additives selected from ionophores, antibiotics, hormones, and mixtures thereof.

19. The feed of claim 12, wherein the caloric contribution consisting of dilution heat plus reaction heat per kilogram of TDDEF contributed by the additive is between 1.5 and 5.0 kcal.

20. The feed of claim 12, wherein the contribution of hydroxyl groups (OH) per kilogram of TDDEF contributed by the additive is between 5.5 and 8.5 grams of OH.

21. The feed of claim 14, which consists of a TDDEF, wherein the pH greater than 9 of the ration with an effective amount of added additive is established with a moisture percentage of 12.5% to 13.5% w/w as a reference.

22. The feed of claim 21, wherein, if the TDDEF has a moisture percentage other than 12.5% to 13.5% w/w, the effective amount of additive to be added to the ration is adjusted taking 12.5% to 13.5% w/w moisture in the ration as a reference value.
